(19) United States
(12) Patent Application Publication
(10) Pub. No.: US 2019/0083147 A1
(43) Pub. Date: Mar. 21, 2019

(54) INSTRUMENT ASSEMBLY FOR USE WITH AN EXPANDABLE PEDICLE SCREW

(71) Applicant: Alphatec Spine, Inc., Carlsbad, CA (US)

(72) Inventor: Thomas Hackathorn, II, Carlsbad, CA (US)

(21) Appl. No.: 15/789,461

(22) Filed: Oct. 20, 2017

Related U.S. Application Data
(63) Continuation-in-part of application No. 15/706,555, filed on Sep. 15, 2017, now Pat. No. 10,172,656.

(51) Int. Cl. A61B 17/70

(52) U.S. Cl. CPC A61B 17/7082; A61B 17/86; A61B 17/7037

(57) ABSTRACT

An instrument assembly for use with an expandable pedicle screw expands the expandable screw while fixing of a distal end of the expandable screw to bone material. The instrument assembly includes a first instrument and a second instrument. The first instrument is configured to screw the expandable pedicle screw into the bone material. The second instrument is configured to slide within the first instrument and fix the distal end of the expandable pedicle screw. The first instrument may engage the expandable pedicle screw and rotate about the second instrument so as to rotate the proximal end of the expandable pedicle screw while the distal end of the pedicle screw remains fixed. Accordingly, the instrument assembly maintains the fixation of the distal end of the expandable pedicle screw with respect to the bone material during the actuation of the expandable pedicle screw.

### CROSS-REFERENCE

This application is a continuation-in-part of U.S. application Ser. No. 15/706,555 filed on Sep. 15, 2017, the contents of which are incorporated herein in their entirety by reference.

### TECHNICAL FIELD

The present disclosure generally relates to an instrument assembly configured to drive and expand an expandable pedicle screw.

### BACKGROUND

Instrument assemblies for use with expandable pedicle screws are known. Such instrument assemblies are configured to both drive the expandable pedicle screw into bone material and to expand the expandable pedicle screw when inserted into the bone material.

Currently, instrument assemblies for expanding an expandable pedicle screw include three instruments. The first instrument is configured to screw the expandable screw into the bone material. The second instrument is configured to slide within a cannula of the first instrument and engage a distal end of the expandable pedicle screw and lock the expandable pedicle screw into place. A third instrument includes a handle that is attached to the second instrument and is configured to twist and pull a distal end towards a proximal end of the expandable pedicle screw so as to expand the expandable pedicle screw. In doing so, a fixation established between the distal end of the expandable pedicle screw and the bone material is deteriorated.

Accordingly, it remains desirable to have an instrument assembly which reduces the number of steps required to expand an expandable pedicle screw and thus reduce surgical time, reduces the number of instruments needed, and eliminates the need for in situ assembly of the instruments. It is further desirable to actuate an expandable surgical screw without disrupting or degrading the fixation established between the distal end of the expandable pedicle screw and the bone material.

### SUMMARY

An instrument assembly for use with an expandable pedicle screw is provided. The instrument assembly is configured to expand the expandable pedicle screw while fixing a distal end of the expandable screw to bone material. Accordingly, the instrument assembly maintains the fixation of the expandable pedicle screw with respect to the bone material during the actuation of the expandable pedicle screw.

The instrument assembly includes a first instrument and a second instrument. The first instrument is configured to rotate the expandable pedicle screw so as to screw the expandable pedicle screw into the bone material. The second instrument is configured to slide within the first instrument so as to fix the distal end of the expandable pedicle screw.

The expandable pedicle screw includes a head and a shaft. The head includes a mating feature configured to receive the first instrument. The shaft includes an expandable section disposed between a distal end and the head. An elongated opening extends through and from the head of the shaft and is closed at the distal end of the shaft. A mating end is disposed within the shaft and at the distal end of the elongated opening.

The first instrument includes a drive shaft and a first handle. The drive shaft includes a cannula defining a throughbore extending between a distal end of the first instrument and a proximal end of the first instrument. A first handle is fixed to a proximal portion of the drive shaft, wherein the proximal end of the cannula is open. A first engagement member is opposite the first handle and disposed at the distal end of the drive shaft. The first engagement member is configured to engage a mating feature disposed on the head of the expandable pedicle screw.

The second instrument includes a fixing shaft and a second handle. The fixing shaft is configured to slide into the cannula of the first instrument. A second engagement member of the fixing shaft fixes the distal end of the expandable pedicle screw. Thus, the distal end may retain its fixation with the bone material while the proximal end of the expandable pedicle screw is threaded further into the bone material. In such a manner, an expandable section of the expandable pedicle screw may be actuated without compromising the fixation achieved by the distal end of the expandable pedicle screw.

Further, the instrument assembly reduces the number of steps required to expand the expandable pedicle screw, reduces the surgical time and the number of instruments needed, and eliminates the need for in situ assembly of the instruments.

### BRIEF DESCRIPTION OF THE DRAWINGS

The embodiments set forth in the drawings are illustrative and exemplary in nature and not intended to limit the subject matter defined by the claims. The following description of the illustrative embodiments can be understood when read in conjunction with the following drawings where like structure is indicated with like reference numerals and in which:

FIG. 1 schematically depicts an instrument assembly in accordance with one or more embodiments described and illustrated herein;

FIG. 2 is an isolated view of a first instrument in accordance with one or more embodiments described and illustrated herein;

FIG. 3 is an isolated view of second instrument in accordance with one or more embodiments described and illustrated herein;

FIG. 4 is an isolated view of an expandable pedicle screw;

FIG. 5A is a cross-sectional view showing the instrument assembly and expandable pedicle screw threaded into bone material; and

FIG. 5B is a view of FIG. 5Aa showing the instrument assembly actuating the expandable screw.

### DETAILED DESCRIPTION OF THE PREFERRED EMBODIMENTS

An instrument assembly for use with an expandable pedicle screw is provided. The instrument assembly is configured to expand an expandable pedicle screw while fixing a distal end of the expandable pedicle screw to bone material. The instrument assembly includes a first instrument and a second instrument. The first instrument is configured to rotate the expandable pedicle screw so as to screw the expandable pedicle screw into the bone material. The second instrument is configured to slide within the first instrument so as to fix the distal end of the expandable pedicle screw. As used herein, the terms “fix” and “fixing”, as used in as a verb or a noun as the case may be, refer to the absence of rotation and proximal movement of the distal end of the expandable pedicle screw relative to the proximal end of the expandable pedicle screw. For example a fixed distal end of the expandable pedicle screw does move in the proximal direction and separate from bone material during expansion of the expandable pedicle screw. The first instrument may engage the expandable pedicle screw and rotate about the second instrument so as to rotate a proximal end of the expandable pedicle screw while the distal end of the pedicle screw remains fixed. The term “distal” as used herein refers to an end of the referenced part which is configured to enter or approach bone material in a surgical procedure. The term “proximal” refers to an end of the referenced part opposite the distal end.

Accordingly, the instrument assembly maintains the fixation of the distal end of the expandable pedicle screw with respect to the bone material during the actuation of the expandable pedicle screw.

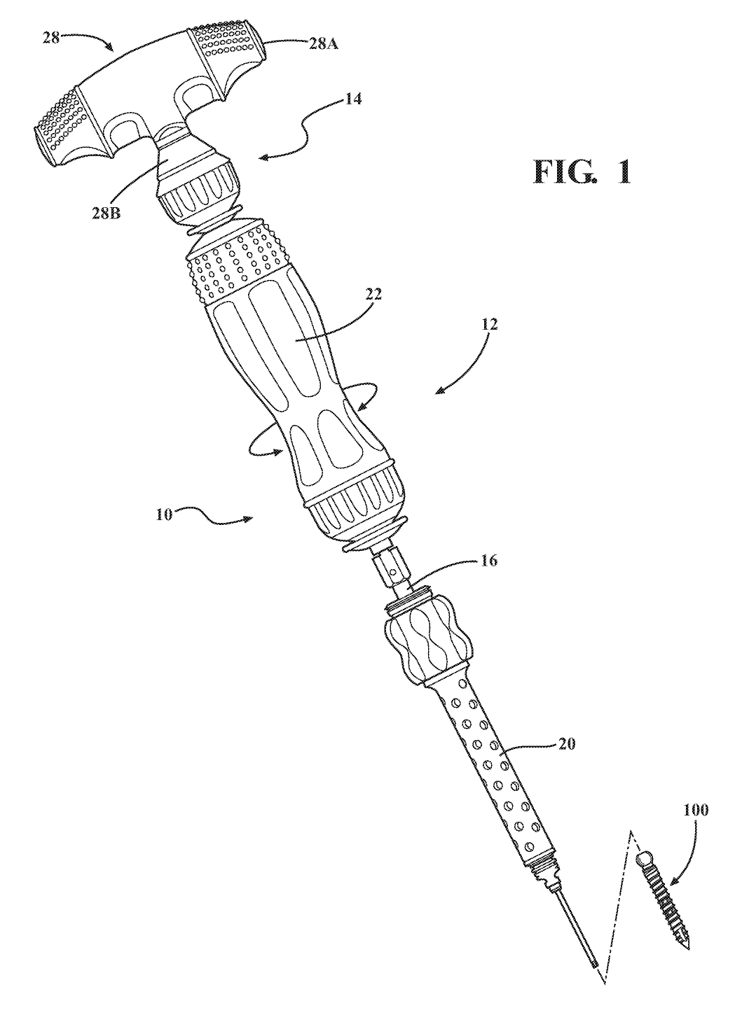

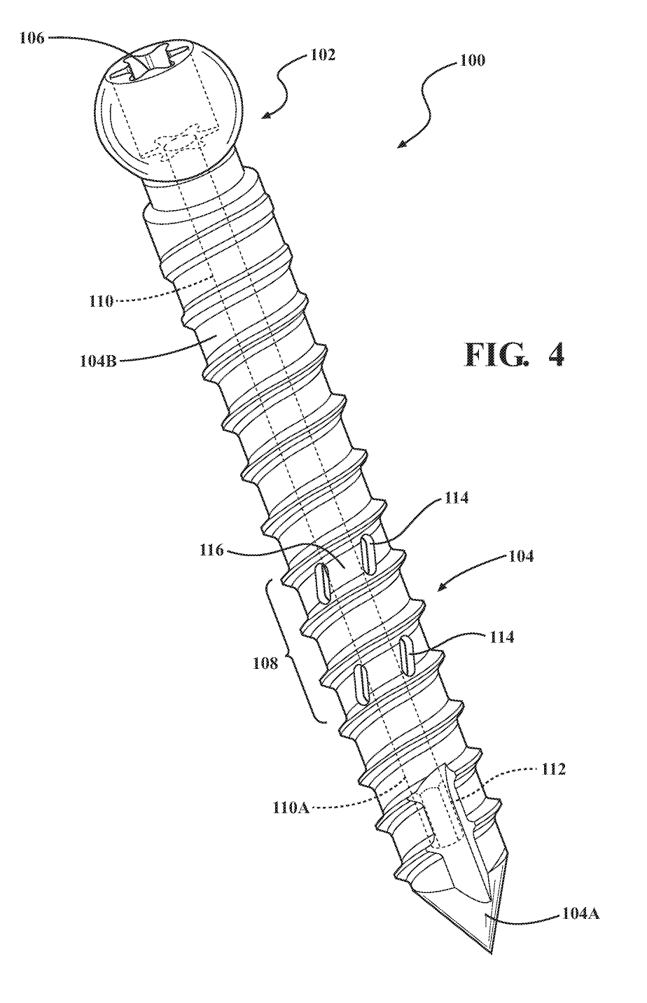

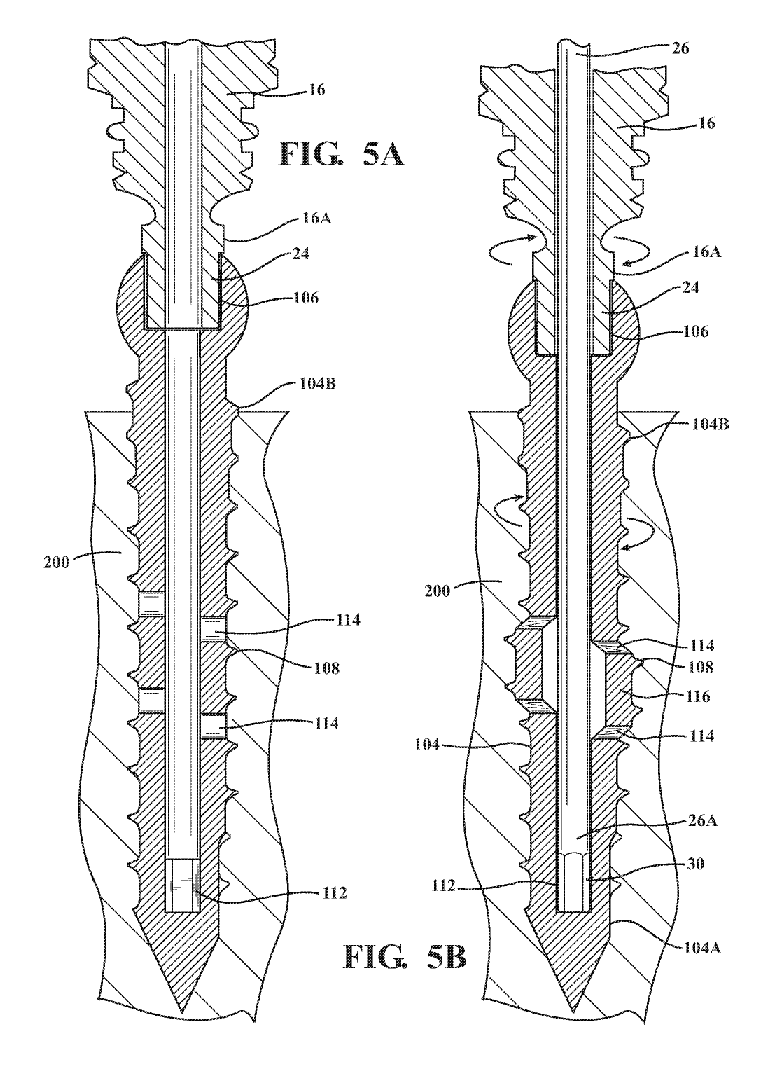

With reference now to FIGS. 1-3 an illustrative view of an instrument assembly 10 is provided. The instrument assembly 10 includes a first instrument 12 configured to rotate an expandable pedicle screw 100 so as to screw the expandable pedicle screw 100 (shown in FIG. 4) into bone material 200 (shown in FIGS. 5A and 5B). The instrument assembly 10 further includes a second instrument 14. The second instrument 14 is configured to slide within the first instrument 12 so as to fix the distal end 104A of the expandable pedicle screw 100 while the first instrument 10 rotates the proximal end 104B of the expandable pedicle screw.

With reference now to FIG. 4, an illustrative depiction of an expandable pedicle screw 100 is provided. The expandable pedicle screw 100 includes a head 102 and a shaft 104. The shaft 104 is threaded. Preferably, the expandable pedicle screw 100 is formed as a single piece. The head 102 includes a mating feature 106 configured to receive the first instrument 12. The shaft 104 includes an expandable section 108 disposed between the distal end 104A of the shaft 104 and the head 102. An elongated opening 110 extends through and from the head 102 of the shaft 104 towards the distal end 104A of the shaft 104 along the axial length of the shaft 104. The elongated opening 110 is bound by the shaft 104, open at the head 102 and is closed by the distal end 104A of the shaft 104. A mating end 112 is disposed within the shaft 104 and at a distal end 110A of the elongated opening 110.

The expandable section 108 is illustratively shown as having a plurality of elongated pores 114 defining a plurality of legs 116. The elongated pores 114 expand the legs 116 radially when the head 102 and the distal end 104A of the expandable pedicle screw 100 are compressed together. It should be appreciated that the expandable pedicle screw 100 depicted and described herein is provided for illustrative purposes and is not limiting to the scope of the appended claims. Any expandable pedicle screw 100 currently known or used which includes an expandable section 108 configured to radially expand under compression may be adapted for use herein.

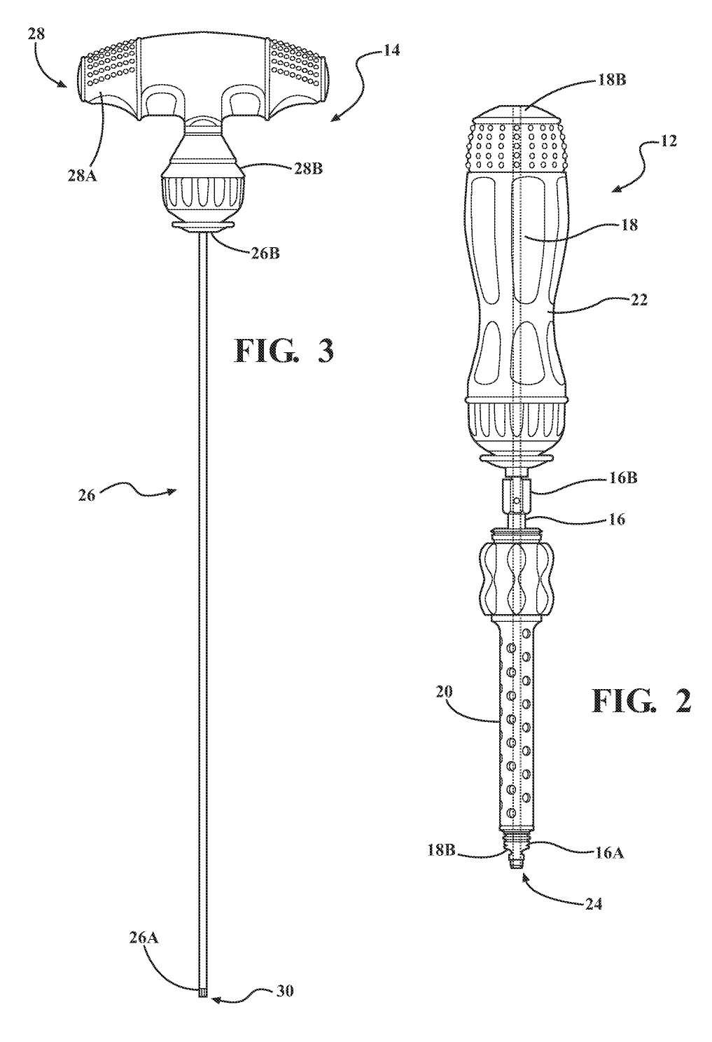

With reference now to FIGS. 1 and 2, a description of the first instrument 12 is provided. The first instrument 12 functions similar to currently known and used polyaxial screwdrivers in that the first instrument 12 is configured to engage a pedicle screw or an expandable pedicle screw 100 and rotate or screw the pedicle screw/expandable pedicle screw 100 into the bone material 200. The first instrument 12 is a generally elongated cylindrical member having a drive shaft 16.

The drive shaft 16 may be formed of a durable and rigid material configured and approved for use in posterior fixation procedures. Such material is currently known and used and illustratively includes medical grade titanium. The drive shaft 16 includes cannula 18 with a diameter ‘D1’. The cannula 18 is open at a proximal end 16B of the drive shaft 16 and a distal end 16A of drive shaft 16 so as to define a throughbore. A sleeve 20 may be mounted on the drive shaft 16. The sleeve 20 is concentric to the drive shaft 16 and may be used to help reduce disruption to surrounding tissue when inserting a screw into bone material during the spinal procedure.

The first instrument 12 includes a first handle 22. The first handle 22 is fixed to the proximal end 16B of the drive shaft 16, wherein rotation of the first handle 22 rotates the drive shaft 16. A proximal end 18B of the cannula 18 is open to the first handle 22. The first handle 22 is configured to facilitate gripping the drive shaft 16 so as to rotate the first instrument 12. In one aspect, the first handle 22 is generally peanut shaped so as to be easily gripped by a user. Frictional features may be made on the first handle 22 to facilitate the grip. The first handle 22 may be formed of a resilient material illustratively including polypropylene.

The drive shaft 16 may further include a first engagement member 24. The first engagement member 24 is opposite the first handle 22 and disposed at the distal end 16A of the drive shaft 16. The first engagement member 24 is configured to engage the mating feature 106 disposed on the head 102 of the expandable pedicle screw 100. For illustrative purposes, the first engagement member 24 is shown as having a hexagonal cross-section and the mating feature 106 of the expandable pedicle screw 100 is a recessed opening having a corresponding hexagonal cross-section so to form a fitted engagement with the first engagement member 24.

With reference now to FIGS. 1 and 3 a description of the second instrument 14 is provided. The second instrument 14 is configured to slide into the cannula 18 of the first instrument 12. The second instrument 14 includes a fixing shaft 26 and a second handle 28. The second handle 28 is fixed to a proximal end 26B of the fixing shaft 26, wherein rotation of the second handle 28 rotates the fixing shaft 26. A diameter “D2” of the fixing shaft 26 is smaller than the diameter “D1” of the cannula 18 so as to allow the drive shaft 16 to freely rotate about the fixing shaft 26. The distal end 26A of the fixing shaft 26 includes a second engagement end 30. The second engagement end 30 is configured to couple with a mating end 112 disposed within the elongated opening 110 of the expandable pedicle screw 100. For illustrative purposes, the second engagement end 30 is shown as having a hexagonal cross-section and the mating end 112 of the expandable pedicle screw 100 has a corresponding hexagonal cross-section so to form a fitted engagement with the second engagement end 30.

The fixing shaft 26 has an axial length longer than the axial length of the drive shaft 16, as shown in FIG. 1. The fixing shaft 26 may be formed of a durable and rigid material configured and approved for use in posterior fixation procedures. Such material is currently known and used and illustratively includes medical grade titanium.

The second handle 28 is illustratively shown as being generally T shaped. The second handle 28 may be formed of a resilient material illustratively including polypropylene. In particular, the second handle 28 includes a gripping portion 28A and a stem 28B. Gripping features may be formed on the underside of the gripping portion 28A of the second handle 28 so as to facilitate the grip of the second instrument 14. The stem 28B may be bulbous so as to help the user retain his or her grip. Thus, the second handle 28 is shaped so as to facilitate an overhand grip, wherein the fingers are placed over the gripping portion 28A and the stem 28B is positioned between the middle finger and ring or index finger as the case may be.

With reference now to FIGS. 5A and 5B an operation of the instrument assembly 10 is provided. FIG. 5A shows the expandable pedicle screw 100 threaded into the bone material 200. The surgeon couples the first instrument 12 with the expandable pedicle screw 100 by inserting the first engagement member 24 with the first mating feature 106. It should be noted that the diameter of the first engagement member 24 is larger than the diameter of the elongated opening 110 so as to maintain the first engagement member 24 within the head 102 of the expandable pedicle screw 100. The surgeon may simply grab the first handle 22 of the first instrument 12 and rotate the first instrument 12 so as to thread the expandable pedicle screw 100 into the bone material 200 a desired depth as shown in FIG. 5A and the distal end 104A of the expandable pedicle screw 100 has found purchase within the bone material 200.

With reference now to FIG. 5B, the second instrument 14 is inserted into the cannula 18 of the drive shaft 16. It should be appreciated that the second instrument 14 may be inserted into the cannula 18 of the drive shaft 16 before the first instrument 12 begins screwing the expandable pedicle screw 100 or after the expandable pedicle screw 100 has been screwed into the bone material 200 a desired depth. As the axial length of the fixing shaft 26 is longer than the axial length of the drive shaft 16, the second engagement end 30 is positioned within the bore of the expandable pedicle screw 100. In particular, the axial length of the fixing shaft 26 is sufficient such that the second engagement end 30 of the fixing shaft 26 engages with the mating end 112 of the expandable pedicle screw 100. In such a manner, the distal end 104A of the expandable pedicle screw 100 is fixed in position.

The user may then grip the second handle 28 with one hand and rotate the first handle 22 of the first instrument 12 with the other. As such, the distal end 104A of the shaft 104 of the expandable pedicle screw 100 is fixed in place by the coupling of the second engagement end 30 of the fixing shaft 26 with the mating end 112 of the expandable pedicle screw 100. Simultaneously the first engagement end of the first instrument 12 is coupled with the mating feature 106 of the head 102, rotating the proximal end 104B of the shaft 104 relative to the fixed distal end 104A of the shaft 104 and expanding the expandable section 108.

FIG. 5B shows the proximal end 104B of the shaft 104 of the expandable pedicle screw 100 threaded deeper into the bone material 200 relative to FIG. 5A. The compression of the expandable pedicle screw 100, that is the advancement of the proximal end 104B of the expandable pedicle screw 100 towards the fixed distal end 104A of the expandable pedicle screw 100, actuates the expandable section 108. FIG. 5B shows the legs 116 of the expandable pedicle screw 100 splayed radially as a result of the compression, wherein the diameter of the shaft 104 of the expandable pedicle screw 100 is increased along the expandable section 108. In such a manner, the fixation achieved by the distal end 104A of the shaft 104 of the pedicle screw with respect to the bone material 200 is not altered or damaged as the distal end 104A remains fixed, yet actuation of the expandable section 108 of the expandable pedicle screw 100 is achieved.

While the invention has been described in connection with various embodiments, it will be understood that the invention is capable of further modifications. This application is intended to cover any variations, uses, or adaptations of the invention following, in general, the principles of the invention, and including such departures from the present disclosure as, within the known and customary practice within the art to which the invention pertains.

I claim:

1. An instrument assembly for use with an expandable pedicle screw, the expandable pedicle screw configured to be threaded into a bone material, the expandable pedicle screw having a head and a shaft, the shaft having a distal end and an expandable section disposed between the distal end and the head, the shaft further including an elongated opening, a mating feature disposed on the head, and an elongated opening extending from the head of the shaft and closed by a distal end of the shaft, wherein a mating end is disposed within the shaft and at the distal end of the elongated opening, the instrument assembly comprising:
a first instrument including a drive shaft having a cannula open at a proximal end of the drive shaft and a distal end of drive shaft, and a first engagement feature configured to couple with the mating feature of the head of the expandable pedicle screw;
a second instrument having a fixing shaft configured to slide within the cannula of the drive shaft, wherein the second instrument includes a second engagement end disposed on a distal end of the fixing shaft, the second engagement member configured to couple with the mating end of the expandable pedicle screw, wherein the first instrument is configured to rotate the expandable pedicle screw so as to screw the expandable pedicle screw into the bone material, and wherein the second instrument is configured to fix the distal end of the expandable pedicle screw with respect to the bone material after the expandable pedicle screw is threaded into the bone material, wherein the first instrument may engage the expandable pedicle screw and rotate about the second instrument so as to rotate the proximal end of the expandable pedicle screw and actuate the expandable section while the distal end of the pedicle screw remains fixed with respect to the bone material.

2. The instrument assembly as set forth in claim 1, wherein the first instrument further includes a first handle and the second instrument includes a second handle.

3. The instrument assembly as set forth in claim 1, wherein the fixing shaft is longer than the drive shaft.

4. The instrument assembly as set forth in claim 1, wherein a diameter of the fixing shaft is smaller than a diameter of the cannula.

5. The instrument assembly as set forth in claim 1, wherein the first instrument further includes a sleeve mounted on the drive shaft and the sleeve is concentric to the drive shaft.

6. The instrument assembly as set forth in claim 1, wherein the first engagement member has a hexagonal cross-section and the mating feature of the expandable pedicle screw is a recessed opening having a corresponding hexagonal cross-section so as to form a fitted engagement with the first engagement member.

7. The instrument assembly as set forth in claim 6, wherein the second engagement end has a hexagonal cross-section and the mating end of the expandable pedicle screw has a corresponding hexagonal cross-section so to form a fitted engagement with the second engagement end.

8. The instrument assembly as set forth in claim 7, wherein the diameter of the first engagement end is larger than the diameter of the elongated opening of the expandable pedicle screw.

9. An instrument assembly for use with an expandable pedicle screw having a head and a shaft, the shaft having a distal end and an expandable section, the shaft further including an elongated opening extending through the head, the expandable pedicle screw configured to be threaded into a bone material, the instrument assembly comprising:
a first instrument including a drive shaft having a cannula open at a proximal end of the drive shaft and a distal end of drive shaft, and a first engagement feature configured to couple with the head of the expandable pedicle screw;
a second instrument having a fixing shaft configured to slide within the cannula of the drive shaft, wherein the second instrument includes a second engagement end disposed on a distal end of the fixing shaft, the second engagement member configured to couple with the distal end of the expandable pedicle screw;
wherein the second instrument is configured to fix the distal end of the expandable pedicle screw while the first instrument simultaneously rotates the head of the expandable pedicle screw so as to actuate the expandable section while the distal end of the pedicle screw remains fixed with respect to the bone material.

10. The instrument assembly as set forth in claim 9, wherein the first instrument further includes a first handle and the second instrument includes a second handle.

11. The instrument assembly as set forth in claim 9, wherein the fixing shaft is longer than the drive shaft.

12. The instrument assembly as set forth in claim 9, wherein a diameter of the fixing shaft is smaller than a diameter of the cannula.

13. The instrument assembly as set forth in claim 9, wherein the first instrument further includes a sleeve mounted on the drive shaft, the sleeve is concentric to the drive shaft.

14. The instrument assembly as set forth in claim 9, wherein the first engagement member has a hexagonal cross-section and the mating feature of the expandable pedicle screw is a recessed opening having a corresponding hexagonal cross-section so to form a fitted engagement with the first engagement member.

15. The instrument assembly as set forth in claim 14, wherein the second engagement end has a hexagonal cross-section and the mating end of the expandable pedicle screw has a corresponding hexagonal cross-section so to form a fitted engagement with the second engagement end.

16. The instrument assembly as set forth in claim 15, wherein the diameter of the first engagement end is larger than the diameter of the elongated opening of the expandable pedicle screw.
